(19) United States
(12) Patent Application Publication
(10) Pub. No.: US 2008/0234540 A1
(43) Pub. Date: Sep. 25, 2008

(54) Osteochondral Implant Procedure

(75) Inventors: Jeffrey H. Nycz, Collierville, TN (US); Susan J. Drapeau, Cordova, TN (US); Daniel Shimko, Germantown, TN (US)

Correspondence Address: HAYNES AND BOONE, LLP, 901 Main Street, Suite 3100, Dallas, TX, 75202, US

(73) Assignee: Warsaw Orthopedic, Inc., Warsaw, IN (US)

(21) Appl. No.: 11/669,888

(22) Filed: Jan. 31, 2007

(51) Int. Cl. A61F 2/04; A61F 5/00

(52) U.S. Cl. 600/36

(57) ABSTRACT

A surgical procedure according to which a moldable material is positioned over a defect and over an area of the anatomy surrounding the defect so that the moldable material takes the shape of the area. After the moldable material forms a mold, a graft is placed on the mold; and cut to conform to a dimension of the defect.

### BACKGROUND

This invention relates to an improved osteochondral implant procedure, and more particularly, to such a procedure and device in which a recipient opening is prepared for receiving a graft.

In the human body, the knee consists of three condyles—a femur, a tibia, and a patella—that are held in place by various ligaments. The corresponding chondral areas of the femur and the tibia form a hinge joint, and the patella protects the joint. Portions of the latter areas, as well as the underside of the patella, are covered with an articular cartilage, which allow the femur and the tibia to smoothly glide against each other without causing damage.

The articular cartilage often tears, usually due to traumatic injury (often seen in athletics) and degenerative processes (seen in older patients). This tearing does not heal well due to the lack of nerves, blood vessels and lymphatic systems; and the resultant knee pain, swelling and limited motion of the condyle(s) must be addressed.

Damaged adult cartilages have historically been treated by a variety of surgical interventions including lavage, arthroscopic debridement, and repair stimulation, all of which provide less than optimum results.

Another known treatment involves removal and replacement of the damaged cartilage with a prosthetic device. However, the known artificial prostheses have largely been unsuccessful since they are deficient in the elastic, and therefore in the shock-absorbing, properties characteristic of the cartilage. Moreover, the known artificial devices have not proven able to withstand the forces inherent to routine knee joint function.

In an attempt to overcome the problems associated with the above techniques, osteochondral transplantation, also known as “mosaicplasty” has been used to repair articular cartilages. This procedure involves removing injured tissue from the damaged area and drilling one or more openings in the underlying condyle. A corresponding number of grafts, or plugs, each consisting of a section of healthy cartilage overlying a corresponding section of the femur, is obtained from another area of the patient, typically from a lower weight-bearing region of the joint under repair, or from a donor patient, and is implanted in the opening. In order to insure a precise fit between the graft and the opening, it is important that the graft has essentially the same dimensions and shape as the opening. However, this is difficult, especially since the outer surface of the condyle and the cartilage are curved.

An embodiment of the present invention involves a technique for insuring that the graft to be implanted has essentially the same dimensions and shape as the opening.

### BRIEF DESCRIPTION OF THE DRAWING

FIG. 1 is an elevational view of a human knee with certain parts removed in the interest of clarity.

FIGS. 2 is an enlarged, partial-sectional, partial-elevational view of the femur of FIG. 1 showing a step in the implanting technique according to an embodiment of the invention.

FIG. 3 is a cross-sectional view of a mold obtained in the step of FIG. 2.

FIG. 4 is a view similar to that of FIG. 3 but depicting a graft disposed on the mold of FIG. 3

FIG. 5 is a view similar to that of FIG. 2 but depicting the graft of FIG. 4 implanted in the femur of FIG. 2.

### DETAILED DESCRIPTION

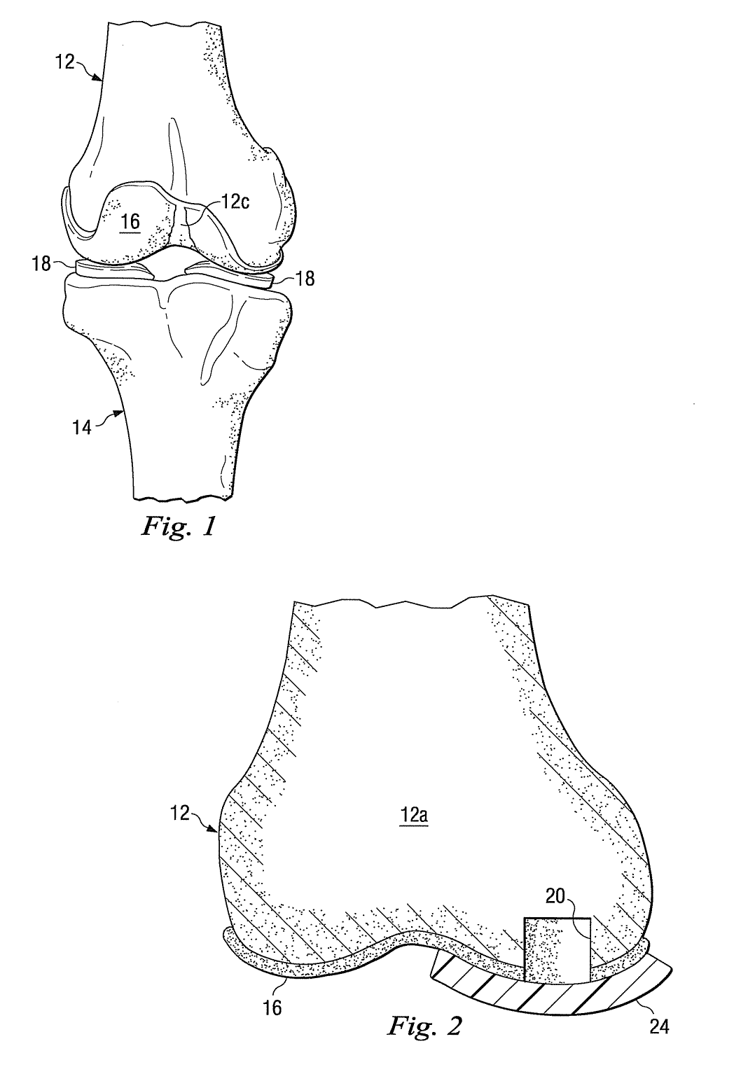

Referring to FIG. 1 of the drawing, the reference numeral 10 refers, in general, to a knee area of a human including a femur 12 and a tibia 14 whose respective chondral areas are in close proximity. A cartilage 16 extends over a portion of the femur 12, and a meniscus 18 extends between the cartilage and the tibia 14. The patella, as well as the tendons, ligaments, and quadriceps that also form part of the knee, are not shown in the interest of clarity.

Referring to FIG. 2, it will be assumed that a portion of the cartilage 16 as well as the underlying portion of the condyle of the femur 12 has been damaged or has worn away, and that the surgeon has shaped the damaged area to form a defect 20 that consists of a void, or opening, that extends through the cartilage 16 and into a portion of the condyle of the femur underlying the cartilage.

A mold 24 is then formed over the defect 20 and over the areas of the cartilage 16 surrounding the defect, with the defect being approximately centered in the mold. The mold 24 can be formed by a relatively soft, fluid or gel-filled pouch that is soft enough so as to take the shape, or contour, of the cartilage 16 surrounding the defect 20 and maintain the shape. For example, the fluid or gel can consist of a reversible polymer or crosslinked polymer that, when placed over the cartilage, will maintain the shape, or contour.

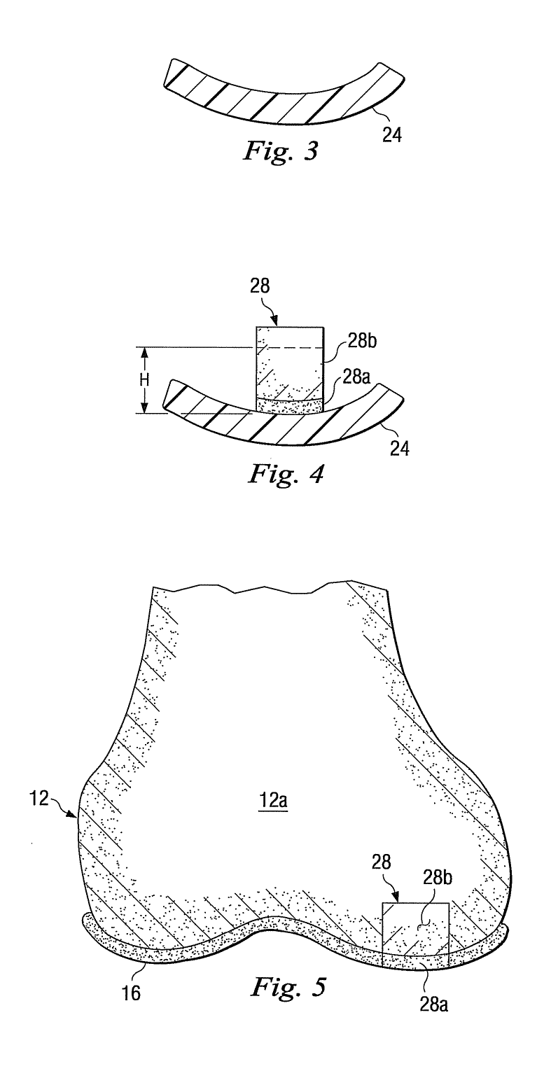

The mold 24 is then removed from the femur 12 and is placed on a table, or the like (not shown), in the position shown in FIG. 3. In this position the mold 24 has a contoured surface 24a that corresponds to the shape of the cartilage 16 and extends upwardly, as viewed in the drawing.

Referring to FIG. 4, a graft 28 that has been harvested from the patient, or from a donor, in any conventional manner is provided. For example the grafting procedure can be of the type disclosed in one or more of assignee's U.S. patent application Ser. Nos. 11/340,024 filed Jan. 26, 2006; Ser. No. 11/338,926 filed Jan. 25, 2006; Ser. No. 11/339,194 filed Jan. 25, 2006; Ser. No. 11/343,156 filed Jan. 30, 2006; Ser. No. 11/339,694 filed Jan. 25, 2006, and (attorney's docket Nos. 31132.552, and 31132.556), the disclosures of which are hereby incorporated by reference.

The graft 28 includes a cartilage section 28a overlying a condyle section 28b. The graft 28 is placed on the center of the upper surface 24a of the mold 24, which corresponds to the area where the defect 20 was located. Since the cartilage section 28a is relatively soft, it will conform to the shape, or contour, of the surface 24a of the mold 24 as shown in FIG. 4.

With the graft 28 properly positioned over the mold 24 as shown in FIG. 4, the graft is cut (in the example shown in FIG. 4, across the dashed line) so that the height H of the graft is identical to the height of the defect 20.

Referring to FIG. 5, the graft 28 is then implanted in the defect 20 using any conventional technique. For example, the implantation procedure can be of the type disclosed in one or more of assignee's U.S. patent application Ser. Nos. 10/792,780, filed on Mar. 5, 2004 (now U.S. publication no. 2004/0176771, published Sep. 9, 2004); Ser. No. 10/785,388, filed on Feb. 23, 2004 (now U.S. application publication no. 2004/0193154, published Sep. 30, 2004); Ser. No. 10/984,497, filed Nov. 9, 2004; (now U.S. application publication no. 2005/0101962, published May 12, 2005); Ser. No. 10/815,778, filed Apr. 2, 2004 (now U.S. application publication no. 2005/0222687, published Oct. 6, 2005); 08/885,752, filed Jun. 30, 1997 (now U.S. Pat. No. 5,919,196 granted Jul. 6, 1999); Ser. No. 08/797,973, filed Feb. 12, 1997 (now U.S. Pat. No. 5,921,987 granted Jul. 13, 1999); Ser. No. 08/908,685, filed Aug. 7, 1997 (now U.S. Pat. No. 5,964,805, granted Oct. 12, 1999); Ser. No. 08/774,799 filed Dec. 30, 1996 (now U.S. Pat. No. 6,007,496); Ser. No. 09/187,283, filed on Nov. 5, 1998 (now U.S. Pat. No. 6,110,209, granted Aug. 29, 2000); Ser. No. 09/425,337, filed Oct. 22, 1999 (now U.S. Pat. No. 6,306,142, granted Oct. 23, 2001); Ser. No. 09/559,532, filed Apr. 28, 2000 (now U.S. Pat. No. 6,375,658, granted Apr. 23, 2002); Ser. No. 09/118,680, filed Jul. 17, 1998 (now U.S. Pat. No. 6,395,011, granted May 28, 2002); Ser. No. 09/624,689, filed Jul. 24, 2000 (now U.S. Pat. No. 6,440,141, granted Aug. 27, 2002); Ser. No. 09/571,363, filed May 15, 2000 (now U.S. Pat. No. 6,488,033, granted Dec. 3, 2002); Ser. No. 09/243,880, filed Feb. 3, 1999 (now U.S. Pat. No. 6,592,588, granted Jul. 15, 2003); Ser. No. 10/004,388, filed Oct. 23, 2001 (now U.S. Pat. No. 6,767,354, granted Jul. 27, 2004); Ser. No. 10/084,490, filed Feb. 28, 2002 (now U.S. Pat. No. 6,852,114, granted Feb. 8, 2005); Ser. No. 10/665,152, filed on Sep. 22, 2003 (now U.S. publication no. 2004/0059425, published Mar. 25, 2004); Ser. No. 10/638,489, filed on Aug. 12, 2003 (now U.S. publication no. 2004/0034437, published Feb. 19, 2004); Ser. No. 10/443,893, filed on May 23, 2003 (now U.S. publication no. 2004/0039400, published Feb. 26, 2004); Ser. No. 10/947,217, filed on Sep. 23, 2004 (now U.S. publication no. 2006/0060209, published Mar. 23, 2006); 11/339,194 filed Jan. 25, 2006; Ser. No. 11/317,985 filed Dec. 23, 2005; Ser. No. 11/340,884 filed Jan. 27, 2006; Ser. No. 11/514,433 filed Sep. 1, 2006; and Ser. No. 11/508,349 filed Aug. 23, 2006. The disclosures of each of these patent applications, publications, and patents are incorporated herein by reference.

In this manner, the contour of the cartilage section 28a of the graft 28 substantially corresponds to the curvature of the area of the cartilage 16 surrounding the original defect 20, and the height of the graft is identical to that of the defect. Thus, the surgeon is assured that the graft 28 has essentially the same dimensions and shape as the original corresponding sections of the cartilage 16 and the condyle of the femur 12 at the defect 20. As a result, the implanted graft 28 will closely simulate the original portion of the cartilage 16 and the femur 12 at the defect 20. Also, the material of the mold 24 can be selected so that it is soft enough to not damage the cartilage section 28a of the graft 28.

### Variations

(1) The location, shape and dimensions of the defect 20 and the graft 28 can vary within the scope of the invention.

(2) The defect 20 and the graft 28 can be in areas of the human anatomy other than the knee.

(3) The type of material(s) forming the mold 24 can vary.

(4) The mold 24 can have a relatively soft upper surface that receives the graft, with the remaining portion of the mold being relatively hard.

(5) The mold 24 can be designed to modulate the shape of a biomaterial placed into an osteochondral lesion. This biomaterial may or may not contain growth factors and/or cells of any origin.

(6) The spatial references mentioned above, such as “upper”, “lower”, “under”, “over”, “between”, “overlying”, “underlying” “above”, and “surrounding” are for the purpose of illustration only and do not limit the specific orientation or location of the components described above.

Those skilled in the art will readily appreciate that many other variations and modifications of the embodiment described above can be made without materially departing from the novel teachings and advantages of this invention. Accordingly, all such variations and modifications are intended to be included within the scope of this invention as defined in the following claims. In the claims, means-plus-function clauses are intended to cover the structures described herein as performing the recited function and not only structural equivalents, but also equivalent structures.

What is claimed is:

1. A surgical procedure for preparing a graft for implantation in a defect in the anatomy of a human, the procedure comprising:
positioning a moldable material over the defect and over an area of the anatomy surrounding the defect so that the moldable material takes the shape of the area;
allowing the moldable material to form a mold;
harvesting a graft;
placing the graft on the mold; and
cutting the graft to conform to a dimension of the defect.

2. The procedure of claim 1 further comprising implanting the graft in the defect.

3. The procedure of claim 1 wherein the area has a contour and wherein a surface of the mold takes the shape of the contour.

4. The procedure of claim 3 wherein that portion of the graft that engages the surface of the mold takes the shape of the latter surface.

5. The procedure of claim 1 wherein that portion of the graft that engages the mold takes the shape of that portion of the mold engaged by the graft.

6. The procedure of claim 1 wherein the step of harvesting comprises harvesting the graft from another area of the patient/recipient, or from a corresponding area of a donor.

7. The procedure of claim 1 wherein the defect is an opening in the femur of the knee of the anatomy.

8. The procedure of claim 7 wherein the defect extends though cartilage overlying the femur.

9. The procedure of claim 7 wherein the upper surface of the femur and the cartilage are curved, and wherein that portion of the mold extending over the defect conforms to the curve.
